(19) United States
(12) Patent Application Publication
(10) Pub. No.: US 2010/0075411 A1
(43) Pub. Date: Mar. 25, 2010

(54) Specimen manipulation device for micro manipulation and biopsy in assisted reproduction and in vitro fertilization

(76) Inventor: Michael D. Cecchi, Madison, CT (US)

Correspondence Address: William W. Jones, 6 Juniper Lane, Madison, CT, 06443, US

(21) Appl. No.: 12/284,316

(22) Filed: Sep. 22, 2008

(51) Int. Cl. C12M 1/00

(52) U.S. Cl. 435/307.1

(57) ABSTRACT

A container or dish used for the micro manipulation, micro injection, biopsy and fertilization of oocyte and embryo culture. The dish allows the user to more readily perform procedures used to fertilize oocyte, such as intracytoplasmic sperm injection (ICSI), biopsy embryos and perform additional procedures in used in assisted reproductive techniques (ART), human reproduction and in vitro fertilization (IVF) techniques. The invention will allow ease in use, the reduction in the number of micro tools used in the procedure, as opposed to conventonal dishes and procedures, and will allow the user to more readily locate the oocytes and embryos to be handled and worked on. The inventon will add repetitiveness, consistency and may result in better results and outcome of the procedures. The invention will also give the user a more ergonomically correct dish for these types of procedures and related protocols.

### TECHNICAL FIELD

This invention relates to a container or Petri type dish that is to be used for the preparation and to allow the user to complete certain procedures on the specimens. The dish allows the user to perform micro manipulations, micro injection and biopsies on the specimens such as oocyte, embryos and stem cells with greater ease, which using fewer micro manipulation tools and may decrease the time and allowing more productivity. This invention relates to the use of multiple impressions in the surface to create a unique working configuration. The configuration allows the ease in locating the specimens. The configuration also provides the ability to perform such multiple procedures, also resulting in the use of fewer micro manipulation tools and instruments, than are now used to perform similar procedures. The dish can be used for a wide range of specimens of animal and human cells, tissues, stem cells, embryos, oocyte, immature oocyte, and uterus slices and while all will receive the same benefits.

### BACKGROUND ART

Current devices, containers or culture dishes used for procedures, such as micro manipulation and are used for intracytoplasmic sperm injection (ICSI), are not unique and are mostly generic, off-the-shelf dishes used for these procedures. The micro manipulation of immature oocytes, oocytes, gametes, zygotes, embryos, cleavage stage embryos, blastocyst stage embryos, and such, all referred to hereinafter as “specimens” are done in standard 50 mm or 60 mm Petri dishes, with no special features, flat bottoms, and lids for storage purposes.

These procedures have become more delicate, need more controls and by creating such specialized devices, containers or dishes will result in increased results and outcome. A major benefit of the invention is higher pregnancy rates from assisted reproductive techniques (ART) and In vitro fertilization (IVF) in the female.

In prior art, there is a dish described as a procedure dish for in vitro fertilization, Bryant, U.S. Pat. No. 6,156,566. This dish is a Petri dish, with a flat surface and etching on the second side to display work places of the dish. This dish does not have any individual wells or intricate and unique configurations of the base, or wells to create a functionality of such as it relates to the procedures.

Problem of the current dishes is that they require the user to use micro drops created on the surface of the dish. Some dishes create surface tension problems with the surface treatment. This results in the micro drops collapsing so that the media solution that the sample is in becomes compromised by an overlaid layer of oil.

Another problem is that the usage of current generic dishes has a tendency to change, as the procedure or protocols are less defined, lack consistency and will vary from procedure to procedure, individual to individual, or day to day. The current dishes require the user performing an ICSI procedure must use a holding pipette and an ICSI pipette. The holding pipette is for holding the oocyte in place and the ICSI pipette is for injection of the sperm into the oocyte for fertilization. This requires the cost of at least two pipettes to perform the procedure and dexterity of the technician performing the procedure. The current off the shelf or flat dishes require the user to locate the specimens, or the single specimen within the droplets. They must spend time to locate the specimen and then grasp the specimen with the holding pipette.

It would be highly desirable to provide a culture dish for in vitro fertilization procedures which will save time and effort in its use. It would also be desirable to provide a culture dish that allows the user to be able to establish repetitiveness and consistency which will help to improve results and improve the outcome of pregnancies and fertility.

### DISCLOSURE OF THE INVENTION

This invention relates to an improved container or dish and a method for using the container or dish for the micro manipulation, micro injection and/or biopsy of oocytes, embryos, and stem cells and other specimens. The dish consists of a plurality of wells, each of which has precisely configured side walls, bases or floors which will hold the specimens in place and allow the user to work on the specimens. The dish will also allow the user to readily locate the specimens therein. The wells are configured to allow the user to readily move about the dish, locate specimens, and perform the procedures more easily. The sizes, shapes and heights of the wells all work together in harmony to allow the user to more effectively perform certain delicate procedures with more confidence and greater results.

The following are several desirable features of the container or dish formed in accordance with this invention.

The improved container or dish of this invention will allow for the micro manipulation or micro injection of embryos, while improving results. The container or dish will allow for the biopsy of embryos and stem cells while improving results.

The dish will also allow the user to reduce costs by reducing the number of instruments and time needed to perform the procedures.

### BRIEF DESCRIPTION OF THE DRAWINGS

These and other objects and advantages of the invention will become more readily apparent from the following detailed description of several embodiments of the invention when taken in conjunction with the accompanying drawings in which:

FIG. 1 is a top plan view of one embodiment of a dish formed in accordance with this invention;

FIG. 2 is a perspective view of a specimen-holding well portion of the dish of FIG. 1;

FIG. 3 is a side sectional view of one of the wells in the dish of FIG. 1 showing a specimen such as an oocyte placed in the well where it can be inseminated;

FIG. 4 is a perspective view of a second embodiment of a dish formed in accordance with this invention;

FIG. 5 is a top plan view of a dish which is operative to separate mobile sperm from immobile sperm; and

FIG. 6 is a cross sectional view of a central portion of the dish of FIG. 11;

### DETAILED DESCRIPTION OF SPECIFIC EMBODIMENTS OF THE INVENTION

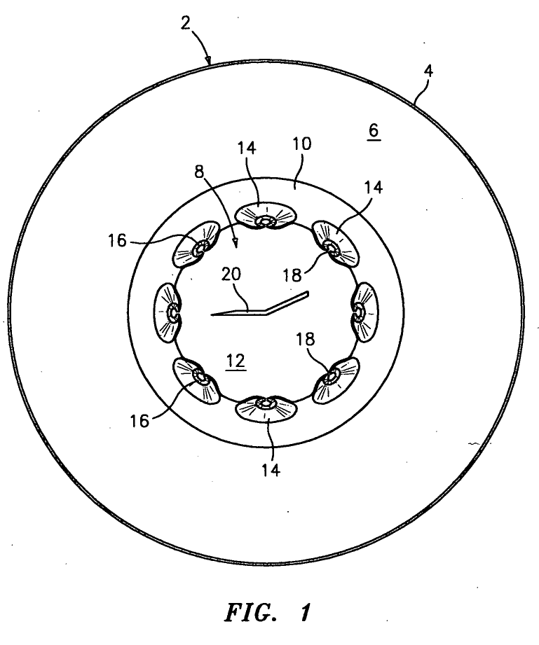

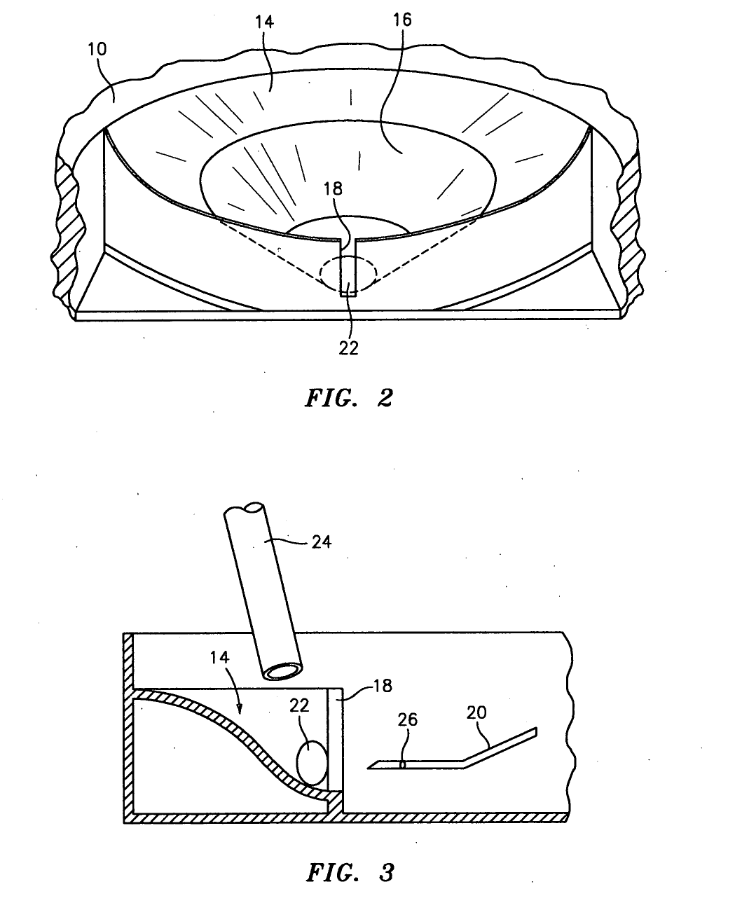

FIG. 1 is a top plan view of one embodiment of a dish 2 formed in accordance with this invention which is designed for the in vito insemination of oocytes. The dish 2 has an outer wall 4 and a bottom wall 6. Within the dish 2 is a well 8. The well 8 has an outer wall 10 and a bottom wall 12. A plurality of chambers 14 are formed on the outer wall 10 of the well 8 with each chamber 14 including an upwardly open pocket 16 disposed therein. Each of the pockets 16 includes an inwardly facing opening 18 in the inner wall thereof. The pockets 16 are for receiving specimens to be treated such as oocytes, embryos, or the like. The openings 18 allow micro tools 20 to enter the pockets 16 to be able to perform an ICSI, biopsy or other procedure on the specimen, which may be an oocyte 22 (See FIG. 2).

FIG. 2 is a front view of one of the chambers 14.shown in FIG. 1. The opening 18 in the chamber 14 has a width of approximately 0.004 Um, which is slightly smaller than the diameter of the average human oocyte 22 and yet is larger than the tip of the micro tool needle 20. It will be noted that the downwardly inclined walls 14 and 16 will force the oocyte 22 or other specimen in the chambers 14 to gravitationally migrate to the lowest point in the chambers 14 and to remain there adjacent to the openings 18 where they can be accessed by the micro tools 20.

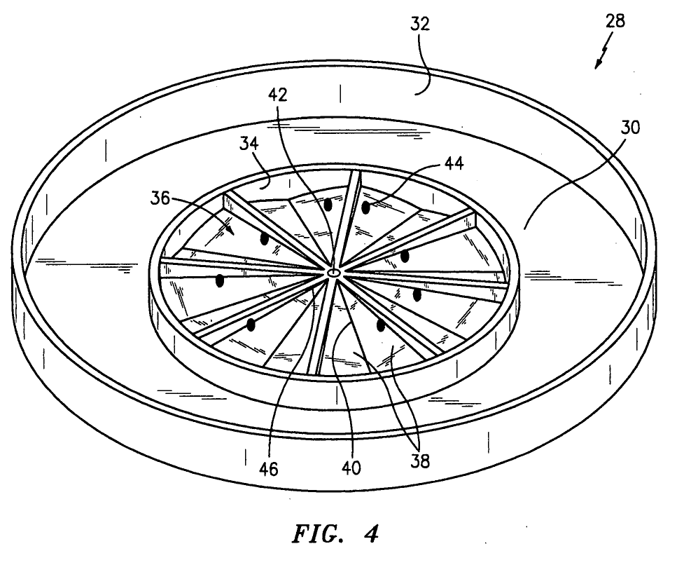

FIG. 3 illustrates the gravitationally induced positioning of an oocyte or embryo 22 in a well 14. The specimen 22 will be introduced into the well 14 by a transfer straw 24. Once properly positioned in the well 14, the specimen 22 is accessible to the micro tool needle 20. If the specimen 22 is an oocyte, the needle 20 can contain a dose of sperm 26 which is to be injected into the oocyte 22 in an attempt to fertilize the latter. It will be noted that only one individual technician is needed to place the oocyte 22 in the chamber 14 and only one individual technician is needed to fertilize the oocyte 22. Furthermore, the technician only needs to use one hand to perform each of the aforesaid procedures. Using the dish 2 shown in FIG. 1, the technician can inseminate eight oocytes from a single donor, which is the typical number of oocytes inseminated in an in vitro fertilization procedure. Once the eight oocytes are inseminated in the dish 2, they will be removed from the dish 2 and placed in an embryo culturing dish of the type shown in FIG. 4.

Referring now to FIG. 4, there is shown a second embodiment of a dish 28 formed in accordance with this invention. The dish 28 includes a floor 30, an outer wall 32, and an inner wall 34. A plurality of wedge-shaped chambers 36 are disposed inside of the inner wall 34. The chambers 36 include downwardly and inwardly angled bottom walls 38 which meet at a central mid line 40. The mid line 40 angles downwardly toward the center 42 of the dish 28. When a biological specimen 44, such as an oocyte, embryo, or the like, is placed in any of the chambers 36, the specimen 44 will gravitationally migrate to the lowest point 46 in the chambers 36, where they will be, in effect, wedged in place.

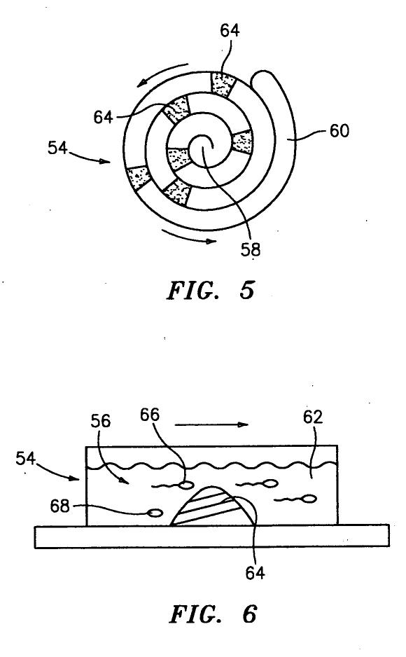

Referring now to FIGS. 5 and 6, there is shown a first embodiment of a dish 54 for separating more motile sperm from less motile sperm so as to provide a more effective sperm supply for use in inseminating oocytes. The dish 54 includes an internal spirally shaped passage 56 which has a central entrance end 58 and an outer exit end 60. The passage 56 is filled with a sperm culturing medium 62 and also includes a plurality of dams 64 which partially block the culturing medium 62. The sperm are injected into the entrance end 58 of the passage 56 by means of a micro tool (not shown). They then swim through the culturing medium 62 toward the outer exit end 60 of the passage 56. While swimming through the passage 56, the sperm encounter the dams 64. Some of the sperm 68 will be blocked by the dams 64 and will not be able reach the outer exit end 60 of the passage 56, while others of the sperm 66 will be able to swim over the dams 64 until they reach the outer exit end 60 of the passage 56. In this manner, the less motile 68 of the sperm are prevented from reaching the outer end 60 of the passage 56 and only the more motile 66 of the sperm can reach the outer end 60 of the passage. This device thus ensures that sperm harvested by the micro tool needle 20 described earlier will be the most motile and healthiest sperm for use in inseminating oocytes.

While the invention has been described with respect to preferred embodiments, those skilled in the art will readily appreciate that various changes and/or modifications can be made to the invention without departing from the spirit or scope of the invention as defined by the appended claims.

What is claimed is:

1. An assembly for use in micro manipulation of embryos, oocytes or other cells, said dish comprising:
a) a dish having at least one compartment for containing a single cell and for fixedly positioning said single cell in a predetermined location in said dish; and
b) a micro tool for micro manipulation of said cell when the latter is in said predetermined location in said dish.

2. The assembly of claim 1 wherein said dish includes a well having an outer side wall, said compartment being created by an upwardly open pocket formed in said outer wall.

3. The assembly of claim 2 wherein said pocket has a downwardly tapered interior in which the cell settles by gravity, said predetermined location being formed by the lowest extremity of said tapered interior of said pocket.

4. The assembly of claim 3 wherein said pocket includes opposite converging side walls formed integrally with said outer side wall of said well, said side walls having opposed end surfaces which are spaced apart from each other to form an opening in said pocket through which said microtool can access a cell disposed in said pocket.

5. The assembly of claim 4 wherein said dish includes a plurality of said pockets disposed on said outer side wall of said well.

6. The assembly of claim 5 wherein said micro tool is a tool for artificially insemnating oocytes disposed in said pockets.

7. An dish for use in micro manipulation of embryos, oocytes or other cells, said dish comprising:
a) a well having an outer side wall; and
b) a plurality of upwardly open pockets formed in said outer side wall, said pockets each having a downwardly tapered interior in which a cell settles by gravity to a lowest extremity of said tapered interiors of said pockets, and said pockets each including opposite conferging side walls formed integrally with said outer side wall of said well, said side walls having opposed end surfaces which are spaced apart from each other to form openings in said pockets through which a micro tool can access a cell disposed in each of said pockets.

8. A dish for separating highly motile sperm from less motile sperm, said dish comprising:
a) a passage for containing a sperm sample, said passage having an entrance end and an exit end; and
b) at least one dam disposed in said passage, said dam being operative to hinder movement of less motile sperm and allow passage of highly motile sperm injected into said entrance end of said passage whereby the more highly motile sperm will swim to said exit end of said passage and the less motile sperm will be unable to swim to said exit end of said passage.

9. The dish of claim 8 wherein said passage takes the form of a helix and wherein said entrance end is at a central area of said helix and said exit end is at a circumferential part of said helix.
